(19) United States
(12) Patent Application Publication
(10) Pub. No.: US 2012/0238492 A1
(43) Pub. Date: Sep. 20, 2012

(54) FISH PROTEIN HYDROLYSATE FOR THE USE THEREOF IN INHIBITING WEIGHT GAIN AND/OR WEIGHT LOSS

(76) Inventors: Elisa Courois, Saint-Malo (FR); Hubert Drieu La Rochelle, Saint-Malo (FR)

(21) Appl. No.: 13/380,472

(22) PCT Filed: Jun. 25, 2010
(86) PCT No.: PCT/EP2010/059082

§ 371 Date: May 7, 2012

(30) Foreign Application Priority Data
Jun. 26, 2009 (FR) 09/54392

(51) Int. Cl. A61K 38/01; A61P 3/06; A61P 3/04

(52) U.S. Cl. 514/4.8

(57) ABSTRACT

The present invention relates to fish protein hydrolysates for the use thereof in inducing weight loss and in limiting and/or inhibiting weight gain. The invention also relates to such hydrolysates in preventing and/or treating excessive weight and/or health problems linked to excessive weight.

The protein hydrolysates according to the invention are obtained by the enzymatic hydrolysis of at least one protein source selected from among the group comprising the following fish: Micromesistius poutassou, Clupea harengus, Scomber scombrus, Sardina pilchardus, Trisopterus esmarki, Trachurus spp., Gadus morhua, Pollachius virens, Melanogrammus aeglefinus, Coryphaenoides rupestris, fish belonging to the order Siluriformes, said enzymatic hydrolysis being performed by an endopeptidase enzyme derived from Bacillus subtilis.

The present invention concerns fish protein hydrolysates for use thereof in causing weight loss or limiting and/or inhibiting weight gain. The invention also concerns such hydrolysates in preventing and/or treating excessive weight and/or health disorders related to excessive weight.

Fish protein hydrolysates can be obtained chemically or enzymatically. In the first case, the hydrolysis of the proteins is carried out using a strong acid or strong base, under strict pH conditions. These hydrolysis conditions considerably impair the quality of the hydrolysates obtained. In the second case, the hydrolysates are obtained by hydrolysis of proteins using endogenous enzymes or exogenous enzymes. These hydrolysis conditions result in hydrolysates of better quality, in particular with regard to hydrolysis using heterogeneous enzymes that make it possible to control the degree of hydrolysis.

Numerous studies demonstrated that the hydrolysates issuing from co-products and products of fishing obtained enzymatically had advantageous nutritional and functional properties (Quaglia et al. 1987; Shahidi et al. 1995; Liceaga-Gesualdo et al. 1999; Kristinsson and Rasco 2000; Liaset et al. 2003). This is because the enzymatic hydrolysis reaction makes it possible to obtain peptides with varied molecular weights that may have novel biological properties compared with a native protein. For example, studies revealed that fish, crustacean and mollusc protein hydrolysates had anticoagulant properties (Rajapakse et al. 2005), antiproliferative properties for cancerous cell lines (Picot et al. 2006), properties of repairing intestinal epithelial tissue

(Fitzgerald et al. 2005), antioxidant properties (Chuang et al. 2000; Suetsuna et al. 2000; Kim et al. 2001; Suetsuna 2002; Wu et al. 2003; Jun et al. 2004; Suetsuna et al. 2004; Je et al. 2008; Raghavan et al. 2008), immunomodulatory properties (Bogwald et al. 1996; Gildberg et al. 1996; Kotzamanis et al. 2007; Tang et al. 2008), and properties of stimulating the growth of certain cell lines (Ravallec-Plé et al. 2000; Guerard et al. 2001).

Thus; during the past years, numerous pharmaceutical, cosmetic or food industries have been interested in the functional properties of protein hydrolysates of fish or any other sea product.

Excessive weight is today a constant preoccupation. This is because an excessive weight may not only give rise to discomfort in the individual suffering from it but it may also give rise to more serious health problems.

Obesity, for example, is a risk factor in numerous health problems, such as cardiovascular illnesses, metabolic disorders, hypertension or cancers.

The origins of excessive weight are very varied. This phenomenon may for example be behavioural in origin, that is to say related to poor food habits, or hormonal, such as during the menopause in women.

Several solutions are at the present time proposed for treating these problems of overweight. Conventionally, treating or preventing overweight may be achieved by a diet. It is known, however, that such a diet is difficult to follow for a good many subjects and may, when it is unsuitable, give rise to lack of nutriments essential to the functioning of the human body. One alternative is therefore the use of active principles capable of intervening in the mechanisms for limiting weight gain and/or weight loss.

In this context, the researches carried out by the applicant company identified hydrolysates obtained from the enzymatic hydrolysis of a protein source composed of certain fishes having properties of inducing in a consumer a weight loss, or a limitation or an inhibition of weight gain.

The invention also concerns the non-therapeutic use of a fish protein hydrolysate obtained by enzymatic hydrolysis of at least one protein source chosen from the group composed of the following fish: Micromesistius poutassou, Clupea harengus, Scomber scombrus, Sardina pilchardus, Trisopterus esmarki, Trachurus spp., Gadus morhua, Pollachius virens, Melanogrammus aeglefinus, Coryphaenoides rupestris, fish belonging to the order Siluriformes, said enzymatic hydrolysis being performed by an endopeptidase enzyme derived from Bacillus subtilis, for causing weight loss or the limitation or inhibition of weight gain.

According to one embodiment of the invention, the inducing of weight loss and the limitation or inhibition of weight gain include a reduction in the body mass, in particular a reduction in the body fat mass, an improvement in the body fat mass/lean body mass ratio, a reduction in adiposity, an improvement in the body composition, a regulation of the lipid metabolism, and more particularly still a stimulation of lypolysis and/or an inhibition of lypogenesis.

The hydrolysate according to the invention can be used in a slimming diet or a diet for preventing weight gain.

According to one feature of the invention, the said hydrolysate does not cause any limitation in the food intake or any phenomenon of satiety.

According to one embodiment of the invention, the fish protein hydrolysate has:
the following molecular profile distribution: 33% to 39% molecules with a molecular weight of less than 300 Da, 34% to 37% molecules the molecular weight of which is between 300 and 1000 Da, 21% to 24% molecules the molecular weight of which is between 1000 and 3000 Da, 3% to 4% molecules the molecular weight of which is between 3000 and 5000 Da and 1% to 2% molecules the molecular weight of which is between 5000 and 10,000 Da,
a lipid content of less than 1%, by percentage of raw product,
a carbohydrate content of less than 4%, by percentage of raw product,
a protein content above 80%, by percentage of raw product,
a mineral matter content of between 5% and 10%, by percentage of raw product.

It also advantageously has the following amino acid composition: glutamic acid 16.9%, aspartic acid 11.7%, lysine 10%, leucine 8.2%, arginine 6.3%, alanine 6.8%, valine 4.8%, isoleucine 4.4%, glycine 5%, threonine 4.5%, serine 4.4%, tyrosine 3.2%, phenylalanine 3.9%, methionine 2.6%, proline 3.4%, histidine 2%, cystine 1%, tryptophane 0.8%, by percentage by weight with respect to the total weight of amino acids.

Such a hydrolysate and the method of obtaining it is described in patent application no. 08 00753 filed by the applicant company, published under the number FR 2 927 336.

Preferentially, the source of fish proteins comprises the pulp obtained from the fillet of the fish.

Preferentially also, the fish protein hydrolysate is obtained by an obtaining method comprising:
the grinding of at least one source of proteins chosen from the group composed of the fish species Micromesistius poutassou, Clupea harengus, Scomber scombrus, Sardina pilchardus, Trisopterus esmarki, Trachurus spp., Gadus morhua, Pollachius virens, Melanogrammus aeglefinus, Coryphaenoides rupestris, fish belonging to the order Siluriformes, in the presence of water, so as to recover the pulp from the said fish,
the enzymatic hydrolysis of the said protein source at a temperature of between 50° and 75° C., for 1 to 5 hours, after the addition of an endopeptidase enzyme derived from Bacillus subtilis so as to obtain a reaction mixture,
stoppage of the said enzymatic hydrolysis by inactivation of the said enzymes after increasing the temperature of the said reaction mixture to a level not below 70° C., for 8 to 20 minutes,
separation of the protein hydrolysate obtained from the rest of the reaction mixture.

Advantageously, the said enzymatic hydrolysis is carried out in an enzyme/protein source ratio between 0.01 and 2%, preferentially equal to 0.75%, at a hydrolysis temperature of 55° C.

The endopeptidase enzyme derived from Bacillus subtilis is preferentially a metalloendopeptidase, or bacillolysine, belonging to the class EC 3.4.24.28 of the EC classification established by the Enzyme Committee and published by the International Union of Biochemistry and Molecular Biology (IUBMB) in 1992.

According to another aspect of the invention, this also concerns a fish protein hydrolysate obtained by enzymatic hydrolysis of at least one protein source chosen from the group composed of the fish Micromesistius poutassou, Clupea harengus, Scomber scombrus, Sardina pilchardus, Trisopterus esmarki, Trachurus spp., Gadus morhua, Pollachius virens, Melanogrammus aeglefinus, Coryphaenoides rupestris, fish belonging to the order Siluriformes, the said enzymatic hydrolysis being carried out by an endopeptidase enzyme derived from Bacillus subtilis for use thereof in the treatment and/or prevention of excessive weight and/or illnesses related to excessive weight.

According to one embodiment of the invention, the treatment and/or prevention of excessive weight comprises the induction of weight loss, the limitation and/or inhibition of weight gain, in particular reduction in body fat mass, improvement to the body fat mass/lean body mass ratio, the treatment of adiposity, the regulation of lipid metabolism, stimulation of lypolysis and/or inhibition of lypogenesis.

According to one embodiment of the invention, excessive weight is related to obesity, to general metabolism disorders or to weight disorders symptomatic of the menopause.

Since it acts on the weight, it is envisaged that fish protein hydrolysate be also used in the treatment and/or prevention of any illness related to excessive weight. This will for example be a question of cardiovascular illnesses, hypertension, hypercholesterolemia or atherosclerosis.

In these embodiments of the invention, the fish protein hydrolysate is as defined previously. In particular, according to this aspect of the invention, the hydrolysate also does not cause any limitation to food intake or the phenomenon of satiety.

The fish protein hydrolysate as defined previously can thus be used for manufacturing a food supplement, or a pharmaceutical or nutraceutical composition, intended for the treatment and/or prevention of excessive weight and/or illnesses related to excessive weight as stated previously.

The fish protein hydrolysate according to the invention can also be used in any cosmetic slimming or thinning method.

The invention thus also concerns a process, or a method, for non-therapeutic treatment for inducing weight loss, or the limitation or inhibition of weight gain, characterised in that it consists of orally administering a fish protein hydrolysate as defined previously.

Finally, the invention concerns a method of therapeutic treatment and/or prevention for inducing weight loss or the limitation or inhibition of weight gain that is characterised in that it consists of orally administering a fish protein hydrolysate as defined previously.

The features of the invention mentioned above, as well as others, will emerge more clearly from a reading of the following description of an example embodiment, the said example being intended to be illustrative and non-limitative.

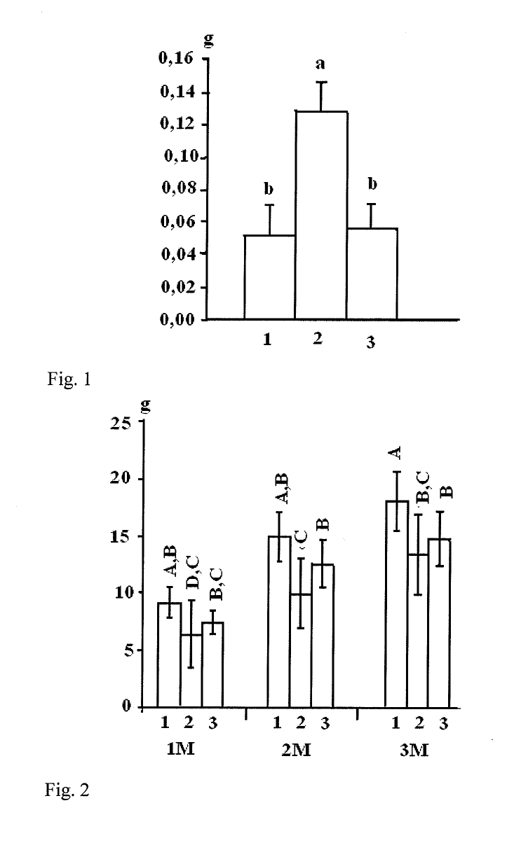

FIG. 1 illustrates the weight, in grams (not referred to 100 g), of the uterus measured in mice at the start of the first study,

FIG. 2 illustrates the total weight gain in grams recorded for each group of mice over three months of the first study,

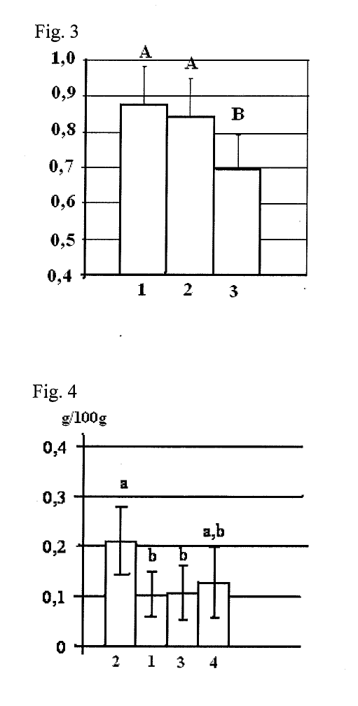

FIG. 3 represents the adiposity indices, that is to say the ratio of the body fat mass to the lean body mass, recorded,

FIG. 4 illustrates the weight of the uterus of the mice in grams for 100 grams of body weight measured at the start of the second study,

FIG. 5 illustrates the total weight gain in grams recorded for each group of mice in the course of the three months of the second study,

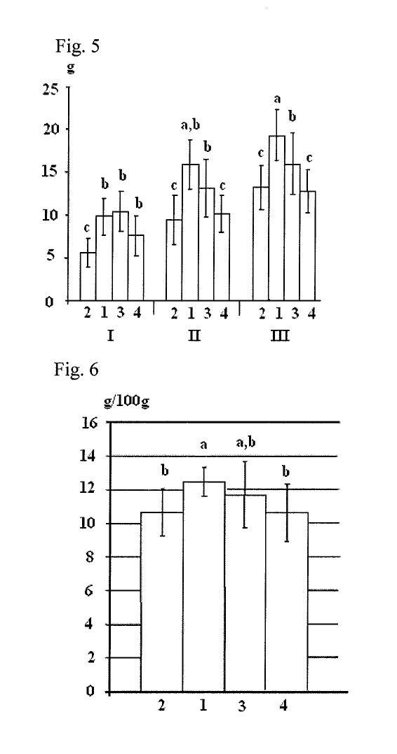

FIG. 6 illustrates the proportion of total subcutaneous adipose tissue for 100 g of body weight, for each group of mice, at the end of the three months of the second study,

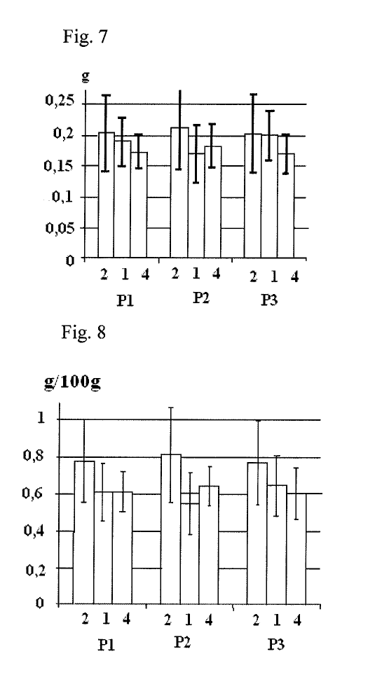

FIG. 7 presents the result of the measurements of the mean meal sizes, expressed in grams, according to the groups, and according to the period studied,

FIG. 8 presents the result of the measurements of the meal sizes expressed in grams for 100 g of body weight, according to the groups, and according to the period studied during the second study,

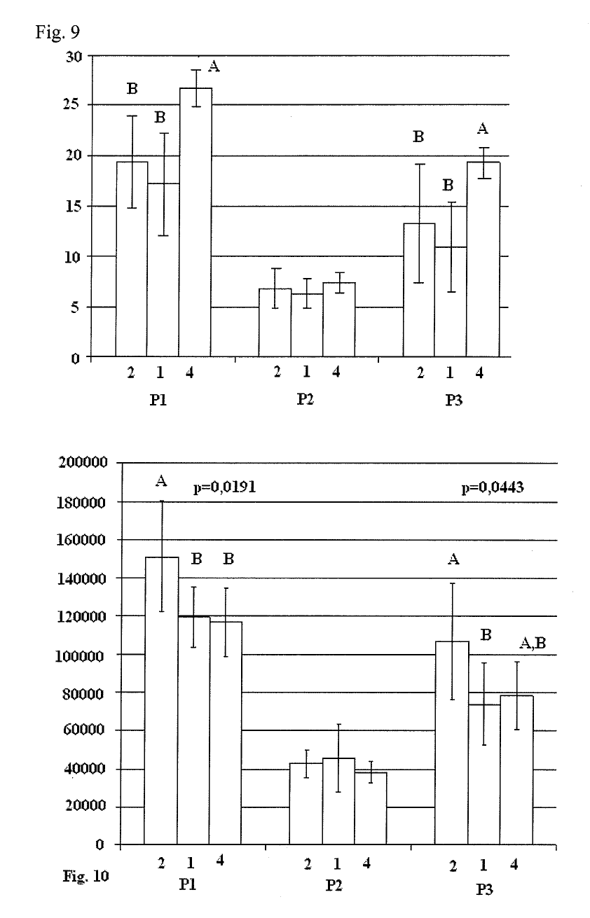

FIG. 9 illustrates the number of meals effected according to the groups and the period studied during the second study, and

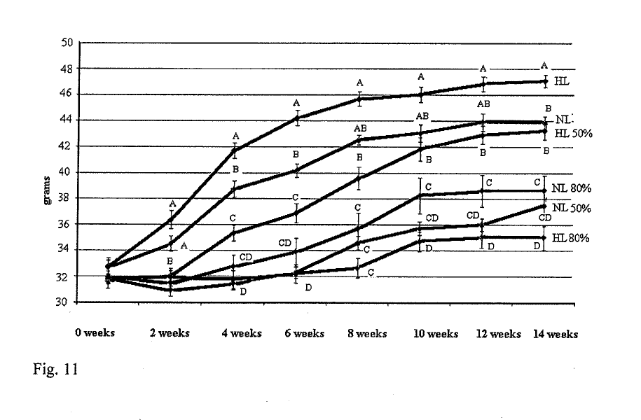

FIG. 10 illustrates the average activity of the mice, expressed in an arbitrary unit, referred to 100 g of body weight according to the groups and the period studied during the second study,

FIG. 11 illustrates the weight gain in grams over time for each group of animals in the course of the third study,

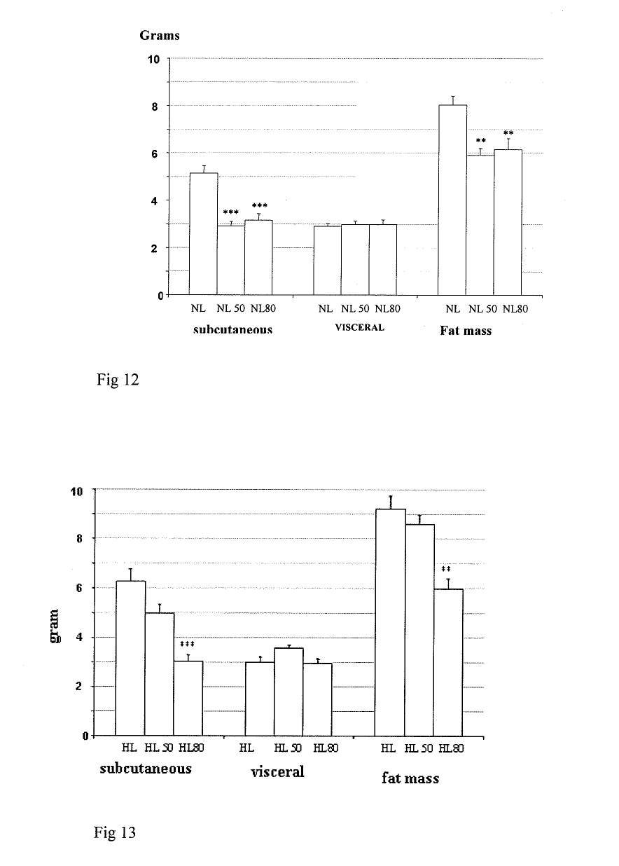

FIG. 12 illustrates the averages of the weights in grams of the subcutaneous tissues and visceral tissues as well as the body fat mass, measured once a week, by MRI, of the mice in group NL during the third study,

FIG. 13 illustrates the averages of the weights in grams of the subcutaneous tissues and visceral tissues as well as the body fat mass, measured once a week during fourteen weeks, by MRI, of the mice in group NL during the third study,

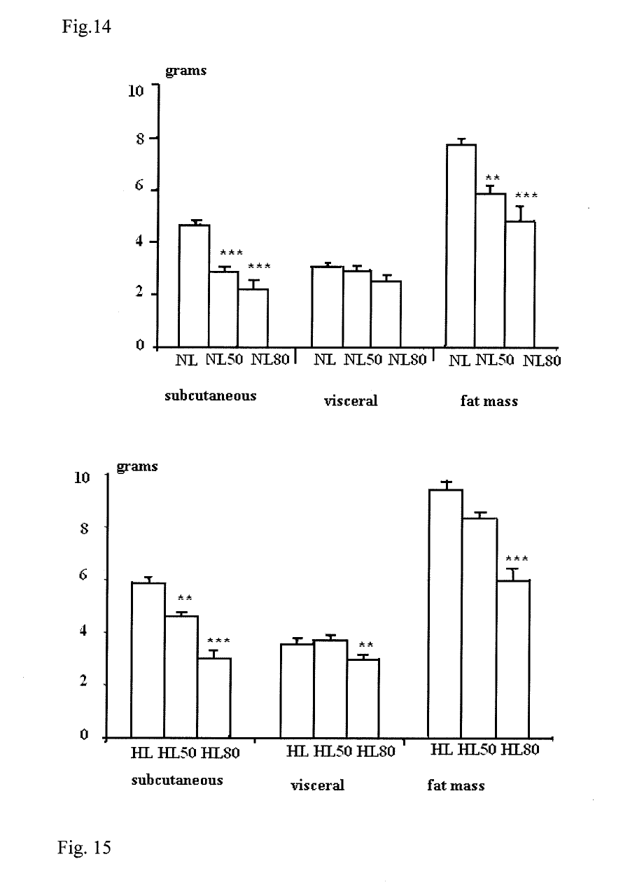

FIG. 14 illustrates the averages of the weights in grams of the subcutaneous tissues and visceral tissues as well as the body fat mass, measured at the end of the study, of the mice in group NL during the third study,

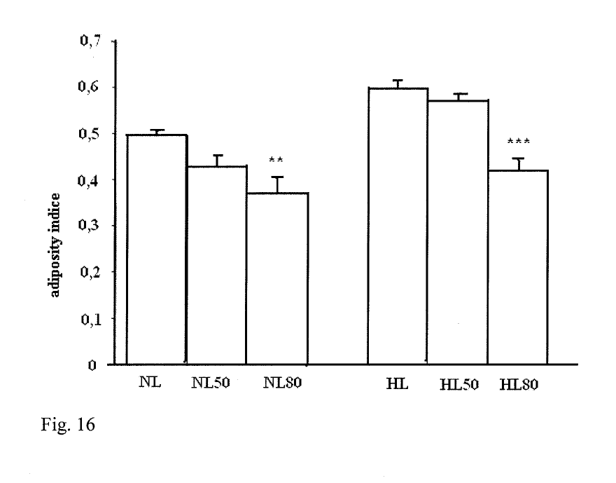

FIG. 15 illustrates the averages of the weights in grams of the subcutaneous tissues and visceral tissues as well as the body fat mass, measured at the end of the study, of the mice in group HL during the third study,

FIG. 16 shows the adiposity indices, that is to say the ratio of the body fat mass to the mass of the carcass, recorded at the end of the study, of the mice in groups HL and NL during the third study,

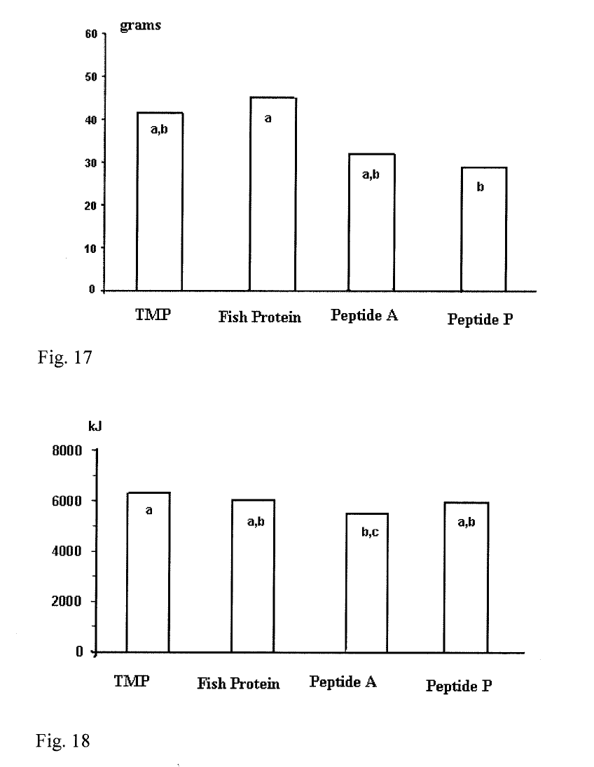

FIG. 17 illustrates the total weight gain in grams recorded for each group of rats during example 5,

FIG. 18 illustrates the total food intake recorded for each group of rats during example 5.

### EXAMPLE 1

### Protein Hydrolysate Obtained From Blue Whiting

Blue whiting (Micromesistius poutassou) is fished in the North Atlantic off Newfoundland. The fish are filleted and the fillets are then ground so as to obtain the pulp therefrom. This fish pulp constitutes a protein source for the production of hydrolysate. The pulp is stored at −20° C. until use.

Three kilos of blue whiting pulp previously defrosted are mixed with water in a weight ratio of 1. The temperature is raised to 55° C. and an endopeptidase enzyme derived from Bacillus subtilis, sold under the name Corolase N by the company AB Enzyme (Feldbergstrafβe 78, D-64293, Darmstadt, Germany) is then added to the reaction mixture in an enzyme/protein source ratio of 0.75%.

The hydrolysis reaction is carried out for 2 hours and then the enzyme is deactivated by raising the temperature of the reaction medium to 85° C. This temperature is maintained for 15 minutes.

The blue whiting protein hydrolysate obtained, hereinafter referred to as H1, is then filtered on a sieve (2 mm/2 mm grille) so as to eliminate the solid matter, and then recovered in a receptacle. The fraction recovered in the receptacle is then centrifuged for 30 plus or minus 5 minutes, at a speed of between 4000 and 7000 rpm. After elimination of the residue, the supernatant is recovered, lyophilised and stored in a cool dry place, away from light. The supernatant can also be atomised.

### EXAMPLE 2

### Protein Hydrolysate Obtained From Other Species of Fish According to the Invention

Hydrolysates of proteins of mackerel (H2) (scomber scombrus), horse mackerel (H3) (Trachurus spp.), grenadier (H4) (Coryphaenoides rupestris), pout (H5) (Trisopterus esmarki), sardine (H6) (Sardina pilchardus), herring (H7) (Clupea harengus), panga (H8) (fish belonging to the order Siluriformes), coalfish (H10) (Pollachius virens), cod (H9) (Gadus morhua) and haddock (H11) (Melanogrammus aeglefinus) were prepared according to the method of example 1.

### EXAMPLE 3

### Physical and Chemical Analyses of the Protein Hydrolysates Obtained According to Examples 1 and 2

A determination of the molecular weights of the peptides constituting each protein hydrolysate H1 to H11 is performed by steric exclusion chromatography (SEC-HPLC).

The protein hydrolysate in powder form after lyophilisation is suspended in ultrapure water at 20 mg/ml, and then filtered over a 0.45 μm membrane and analysed by filtration on gel with a Superdex Peptide HR 10/30 column, sold by the company Pharmacia. The matrix of the column is composed of a crosslinked porous gel (diameter 13-15 μm) of agarose and dextran to a total volume of 24 ml. Its fractionation domain is between 100 and 7000 Da. The column is mounted on an HPLC chain, sold by the company Dionex, which is equipped with a pump (Dionex P680 module). The measurement is performed by a multiwavelength ultraviolet detector (Dionex UVD 170 U module). The protein hydrolysate H1 is eluted by a mobile phase containing acetonitrile, water and TFA. The elution lasts for approximately one hour at a rate of 0.5 ml/min.

The distribution of the molecular weights is calculated from the parameters of a calibration straight line obtained after passing through a column of the following known molecular weight markers: Cytochrome C (12,400 Da), aprotinine (6511 Da), gastrin I (2126 Da), substance P fragment 1-7 (1348 Da), glycine (75 Da) and leupeptine (463 Da). The data are collected by means of Chromeleon software (Dionex). The percentages of the molecular weights are calculated by means of software (GPC Cirrus from Polymer Laboratories). The acquisition wavelength is 214 nm. The distribution of the molecular weights according to dW/logM is given by the software. The percentage of the area under the curve corresponds to the percentage of molecules. The distribution of molecular weight per size class is given in Table 1:

The distribution of the molecular weights per size class is given in the following Table 1. The percentage of the area under the curve corresponds to the percentage of peptide molecules.

All the hydrolysates show an identical distribution profile of the molecular weights.

TABLE 1

| Classes | H1 | H2 | H3 | H4 | H5 | H6 | H7 | H8 | H9 | H10 | H11 |
|---|---|---|---|---|---|---|---|---|---|---|---|
| <0.3 | 33-39 | 39 | 33 | 33 | 33 | 35 | 33 | 33 | 36 | 33 | 34 |
| 0.3-1 | 34-37 | 34 | 37 | 37 | 37 | 37 | 37 | 37 | 37 | 36 | 37 |
| 1-3 | 21-24 | 22 | 24 | 24 | 24 | 22 | 24 | 24 | 22 | 24 | 23 |
| 3-5 | 3-4 | 4 | 4 | 4 | 4 | 4 | 4 | 4 | 3 | 4 | 4 |
| 5->10 | 1-2 | 1 | 2 | 2 | 2 | 2 | 2 | 2 | 2 | 2 | 2 |

The amino acid composition of the protein hydrolysate H1 is given in Table 2 and is obtained in accordance with the indications in European Directive 98/64/CE and the standard NF EN ISO 13904—October 2005.

TABLE 2

| | Amino acid | | Amino acid |
|---|---|---|---|
| Amino acid | percentage | Amino acid | percentage |
| Glutamic acid | 16.9 | Glycine | 5 |
| Lysine | 10 | Threonine | 4.5 |
| Aspartic acid | 11.7 | Serine | 4.4 |
| Leucine | 8.2 | Tyrosine | 3.2 |
| Arginine | 6.3 | Phenylalanine | 3.9 |
| Alanine | 6.8 | Methionine | 2.6 |
| Valine | 4.8 | Proline | 3.4 |
| Isoleucine | 4.4 | Histidine | 2 |
| Cystine | 1 | Tryptophane | 0.8 |

The protein content is above 80%, by percentage of raw product (NF V18-120—March 1997, KJELDAHL corrected).

The lipid content is less than 1%, by percentage of raw product (according to European Directive 98/64/CE).

The energy value of the protein hydrolysate H1 is approximately 350 Kcal/100 g.

The carbohydrate content is less than 4% (derived from the protein and carbohydrate contents and the energy value).

### EXAMPLE 4

### Biological Activities of a Blue Whiting Protein Hydrolysate H1

The protein hydrolysate H1 was tested vis-à-vis its activity on the weight in a mammal.

During following studies, an ovariectomised mouse model was used in order to cause an oestrogen deficiency and to mimic a menopause phenomenon.

Study No. 1:

A diet containing 14% by weight fish protein hydrolysate H1 was compared with a reference standard diet containing 14% by weight of caseins.

Thirty six C3H mice, supplied by the Harlan breeding centre, were placed in cages in a room thermostatically controlled at 22° C. in a reversed day-night cycle (night from 6 a.m. to 6 p.m.). The animals were habituated to their environment and to their food in powder form for 2 weeks. Each animal received water and food ad libitum (5 grams per day and per mouse) during the three months of the study.

At twelve weeks, the mice underwent either an ovariectomy or surgery without ablation of the ovaries. Following the operation, the animals were divided into 3 groups of 12 individuals according to the diets that they were to receive:
Group 1: The mice undergo the ovariectomy and receive the control diet.
Group 2: The mice are operated on without undergoing ablation of the ovaries and receive the control diet, that is to say 14% caseins.
Group 3: The mice undergo the ovariectomy and receive a diet containing 14% H1 (same composition as the control diet but the milk proteins are replaced by the fish protein hydrolysate obtained according to Example 1)

All the diets supplied contain 14% proteins and have similar energy compositions as indicated in Table 3 below.

TABLE 3

| | Constituents in g/kg of | Constituents in g/kg of |
|---|---|---|
| | diet groups 1 and 2 | diet group 3 |
| Total milk proteins g | 140.0 | 0.0 |
| H1 (powder) g | 0.0 | 140.0 |
| Total Kcal/kg of diet | 3617.4 | 3582.8 |

At the end of 3 months of diet, the mice are anaesthetised (injection of 0.1 ml for 10 grams of body weight of a solution of xylazine 1 mg/ml and ketamine 10 mg/ml) and are then sacrificed. The body composition is established by taking and weighing various adipose tissues (periovarian, mesenteric, perirenal, subcutaneous, brown). The weight of the various organs (kidney, liver, uterus, spleen, intestine) is also assessed.

Validation of the Model

At the time of sacrifice, the weights of the uteri were measured precisely in order to verify that the ovariectomy was carried out correctly. In such a case, the uterus must appear to be atrophied. In the contrary case, the mice were excluded from the study. These results are shown in FIG. 1, which reports the weight, in grams (not referred to 100 g), of the measured uterus of the mice. The results showed that the mice in group 2, which did not suffer ablation of the ovaries, had a uterus significantly larger than that of the mice in all the other groups. In FIG. 1, the values are given in the form of an average plus or minus the standard deviation values. The groups that do not have the same letters are statistically different (p<0.05).

Thus no problem was detected during the verification of the efficacy of the ovariectomy.

Weight Gain Over Time:

FIG. 2 illustrates the total weight gain in grams recorded for each group of mice over the course of the three months of the study. The values are given in the form of an average plus or minus the standard deviation values. The groups that do not have the same letters are statistically different (p<0.05). Before the ablation of the ovaries or the operation without ablation of the ovaries, all the groups had a comparable average weight. After one month of diet, differences appeared between the groups, differences that are accentuated over time. Thus, as shown by FIG. 2, after 3 months of diet, the total weight gain observed for the reference group 1 is statistically higher than that reported for groups 2 and 3 (very significant results p<0.001). An “ovariectomy” effect is observed since the mice in group 1 statistically gained more weight than the reference mice in group 2. This effect is not found in the mice in group 3 since they are statistically thinner than the mice in group 1 after three months of diet. This observation confirms that the diet received by group 3 is effective in limiting the weight gain caused by the ovariectomy.

The diet based on hydrolysate H1 limits the weight gain compared with the diet consisting of caseins.

Adiposity Index:

The weights of the carcasses, that is to say the lean body mass, and the adipose masses, were measured for each of the groups at the end of the three months of the study. These two parameters made it possible to calculate the adiposity index (ratio of the body fat mass to the lean body mass) shown in FIG. 3. The adiposity indices are identical with regard to groups 1 and 2 whereas that of group 3 is statistically smaller.

The diet based on hydrolysate H1 causes a reduction in the body fat mass and an increase in the lean body mass, significantly improving the adiposity index.

Study No. 2:

Diets containing 7.5% or 12.5% by weight of fish protein hydrolysates H1 were compared with a reference standard diet containing 14.6% by weight of caseins.

Forty six C3H mice, supplied by the Harlan breeding centre, were placed in cages in a room thermostatically controlled at 22° C. in a reversed day-night cycle (night from 6 a.m. to 6 p.m.). The animals were habituated to their environment and to their food in powder form for 2 weeks. Each animal received water and food ad libitum (5 grams per day and per mouse) during the three months of the study.

At eight weeks, the mice underwent either an ovariectomy or surgery without ablation of the ovaries. Following the operation, the animals were divided into 4 groups of 12 individuals according to the diets that they were to receive:
Group 1: The mice undergo the ovariectomy and receive the control diet.
Group 2: The mice are operated on without undergoing ablation of the ovaries and receive the control diet, that is to say 14.6% caseins.
Group 3: The mice undergo the ovariectomy and receive a diet containing 7.5% H1 (same composition as the control diet but some of the milk proteins are replaced by the fish protein hydrolysate obtained according to Example 1)
Group 4: The mice undergo the ovariectomy and receive a diet containing 12.5% H1 (same composition as the control diet but the milk proteins are replaced by the fish protein hydrolysate obtained according to Example 1)

The energy composition of the diets supplied is indicated in Table 4 below.

TABLE 4

| | Constituents | Constituents | Constituents |
|---|---|---|---|
| | in g/kg of | in g/kg of | in g/kg of |
| | diet groups 1 and 2 | diet group 3 | diet group 4 |
| Caseins g | 146.7 | 60.6 | 0.0 |
| H1 (powder) g | 0.0 | 75.0 | 125.0 |
| Total Kcal/kg of diet | 3721.3 | 3554.5 | 3249.3 |

At the end of 3 months of diet, the mice are anaesthetised (injection of 0.1 ml for 10 grams of body weight of a solution of xylazine 1 mg/ml and ketamine 10 mg/ml) and are then sacrificed. The body composition is established by taking and weighing various adipose tissues (periovarien, mesenteric, perirenal, subcutaneous, brown). The weight of the various organs (kidney, liver, uterus, spleen, intestine) is also assessed.

Validation of the Model

At the time of sacrifice, the weight of the uterus was measured precisely in order to verify that the ovariectomy had been carried out correctly (atrophied uterus) as during study no. 1. These results, shown in FIG. 4 (weight of the uterus in grams for 100 grams of body weight), showed that the mice in group 2, which did not undergo ablation of the ovaries, had a significantly larger uterus than that of the mice in all the other groups. In FIG. 14, the values are given in the form of an average plus or minus the standard deviation values. The groups not having the same letters are statistically different (p<0.05).

No problem was detected during the verification of the validity of the model.

Weight Gain Over Time:

FIG. 5 illustrates the total weight gain in grams recorded for each group of mice over the three months of the study. Times I, II and III correspond respectively to the measurements performed after 1, 2 and 3 months of diet. The values are given in the form of an average plus or minus the standard deviation values. The groups that do not have the same letters are statistically different (p<0.05). Before ablation of the ovaries (groups 1, 3 and 4) or the operation without ablation of the ovaries (group 2), all the groups had a comparable average weight. After three months of diet, significant differences appeared between the groups. Thus the total weight gain observed for reference group 1 of ovariectomised mice is statistically higher than that reported for groups 2, 3 and 4. In addition, the total weight gain observed for group 4 that receives the highest quantity of fish protein hydrolysate is significantly less than the total weight gain observed for group 3. There is no significant difference between the total weight gains recorded for reference group 2 and group 4. These observations demonstrate that the fish protein hydrolysates received by groups 3 and 4 limited a weight gain over time that would be due to the ovariectomy.

Thus, the food diet based on hydrolysate H1 at concentrations 12.5% and 7.5% significantly reduces the weight gain caused by the ovariectomy, and this after two months of diet.

Proportion of Adipose Tissues for 100 g of Body Weight:

FIG. 6 shows the proportion of total subcutaneous adipose tissues for 100 g of body weight, for each group of mice, at the end of three months of study. A significant difference is again observed between the mice in group 4 and the ovariectomised mice in group 1 that did not receive the fish protein hydrolysate. The latter mice have a proportion of subcutaneous adipose tissue greater than that measured for the mice in group 4.

The diets based on hydrolysate H1 therefore have a positive effect on the reduction of adipose tissue.

Measurement of the Food Intake:

A study on the food intake by mice was carried out in order to verify that the effect observed on the weight of the fish protein hydrolysate is not the result of an aversion to the diet, thus giving rise to a loss of weight, or the result of a satiogenic effect of the hydrolysate that would minimise the food intake.

To do this, five mice in group 1, six mice in group 2 and five mice in group 4 are individualised during five full days in metabolic cages. These metabolic cages make it possible to record the quantity of food ingested as a function of time as well as the activity of the mice. The first three days are adaptation days for the mice to this environment. The food intakes and the activities are measured during the last two days. The food is supplied in semi-liquid form during the five days.

Size of the Meals Taken by the Mice:

FIG. 7 presents the result of the measurements of average meal sizes, expressed in grams, according to the groups, and according to the period studied: P1: total period; P2: day; P3: night.

FIG. 8 presents the result of the measurements of the meal sizes expressed in grams for 100 g of body weight, according to the groups, and according to the period studied: P1: total period; P2: day; P3: night.

Number of meals effected according to the groups and the period studied: FIG. 9 illustrates the number of meals effected according to the groups and the period studied: P1: total period; P2: day; P3: night. The groups that do not have the same letters are statistically different (p<0.05).

As is clear from FIGS. 7 and 8, no significant difference is observed between the various groups with regard to the size of the meals. On the other hand, FIG. 9 illustrates significant differences between groups 4 and groups 1 or 2 with regard to the number of meals taken by the mice during the nocturnal period (P3) as well as during the total period (P1). The mice in group 4, the food diet of which includes the fish protein hydrolysate, feed more often than the mice the food diet of which does not include the fish protein hydrolysate.

This study demonstrates that the fish protein hydrolysate does not have a satiogenic effect and also does not cause an aversion to the food diet.

Thus, the group receiving the diet containing H1 has a meal size identical to the other reference groups, but the number of meals is significantly higher. Therefore the group receiving H1 ingests significantly more food but is significantly thinner than the reference group receiving caseins, and this after two months of diet.

Measurement of the Activity of the Animals:

FIG. 10 illustrates the average activity of the mice (arbitrary unit) referred to 100 g of body weight according to the groups and the period studied (P1, P2, P3).

Ovariectomy causes a significant reduction in the total activity, as shown by the significant differences observed between group 1 (ovariectomised) and group 2 (not ovariectomised), which shows greater activity.

Ovariectomy in the mice causes in them a significant reduction in the expenditure of energy.

Conclusions

Thus the fish protein hydrolysate H1 obtained by means of an endopeptidase derived from Bacillus subtilis limited the increase in weight whereas it did not limit the food intake. In addition, ingestion of this hydrolysate is not accompanied by an increase in the physical activity of the mice. Its effect on the weight of the mice is therefore due to one or more physiological phenomena.

### Study No. 3

Seventy two male C3H mice were selected and divided into groups:
3 groups receiving normo-lipidic (NL) diets including 0% (group called NL), 50% (group called NL 50%) or 80% (group called NL 80%) fish protein hydrolysate H1, as a percentage with respect to the total energy contributed by the proteins.
3 groups receiving hyper-lipidic (HL) diets including 0% (group called NL), 50% (group called NL 50%) or 80% (group called NL 80%) fish protein hydrolysate H1, as a percentage with respect to the total energy contributed by the proteins.

The mice in each group are weighed every week and their weight is noted. At the end of fourteen weeks of diet, the mice are sacrificed for analysis of the effect of the hydrolysate H1 on their body composition.

FIG. 11 illustrates the weight gain over time for each group of animals. The values are noted in the form of average in grams plus or minus the standard deviation values. The groups that do not have the same letters are statistically different (p<0.05).

It is observed that the mice fed with the hydrolysate H1 put on less weight than those that did not receive the hydrolysate (groups NL and HL) both in the case of a hyper-lipidic diet and in the case of a normo-lipidic diet. The hydrolysate according to the invention significantly reduced, or limited, the weight gain.

The body composition of the mice was analysed both during and at the end of the experiment. The averages, per group, of the weights in grams of the subcutaneous tissues, the visceral tissues, the body fat mass and the mass of the carcasses are determined and then noted.

The body composition of the mice by magnetic resonance imaging (MRI) was determined immediately after passage through a metabolic cage once a week. The mice were put to sleep with isoflurane using a mask (3% for putting the mice to sleep, reduced to 1% for keeping asleep). The rectal temperature of the mice was measured electronically (SA Instruments) and maintained at 37.0±0.5° C. by means of a heated mattress system. The apparatus took 96 images, which afforded a representation of the animal in 3 dimensions. These images being precise only over 2 cm, the animal had to be moved in order to have an image of the full body. The MIPAV® software used then made it possible to join the photographs in order to reproduce an image of the entire body and to process the data. The adipose tissue, which appeared in white on the photographs, was delimited, and the adipose, subcutaneous and visceral tissues were separated. This treatment was carried out on all the images one by one.

FIGS. 12 and 13 show the weight, in grams, of the various adipose tissues of the mice in groups NL and HL respectively, determined once a week throughout the MRI study. Likewise, FIGS. 14 and 15 illustrate the weights, in grams, of these various adipose tissues taken when the mice are sacrificed. The values are given in the form of average ±SEM. Statistical differences: *p<0.05; **0.0001<p<0.01; ***p<0.0001.

Ingestion of diets enriched with hydrolysate H1 causes a significant reduction in the body fat mass of the mice compared with the reference that did not ingest the hydrolysate H1. This difference is associated with a reduction in subcutaneous adipose tissue (FIGS. 12, 13, 14, 15).

Analyses of the images obtained in MRI show that a significant reduction in the weight of the body fat mass is observed in mice fed with H1 hydrolysate enriched diets (FIGS. 12, 13). In group HL, this significant reduction is observed only in the animals fed with the 80% HL diet. This difference is due solely to a reduction in the mass of the subcutaneous adipose tissue. Enrichment of the diets with H1 hydrolysate has no effect on the visceral adipose tissues of the mice.

The incorporation of hydrolysate H1 in the diet of the mice limited the gain in weight. This limitation in the weight gain is related to the body fat mass, which is statistically less in the groups that received the hydrolysate compared with those that did not receive it.

A significant reduction in the weight of the body fat mass was also observed from the body compositions produced by dissections during sacrifice. This reduction is observed in mice ingesting the 50% H1 hydrolysate diet but is more marked in mice fed with the 80% diet, compared with those ingesting the diets without H1 hydrolysate. This significant reduction in weight is found again with regard to the subcutaneous adipose tissue for the two groups of mice (NL, HL) fed with diets enriched with H1 hydrolysate.

In animals that ingested the 50% HL diet, ingestion did not cause any significant reduction in the weight of the total body fat mass, only a tendency is observed.

However, the weight of the subcutaneous adipose tissue is for its part reduced significantly compared with the control HL diet. On the other hand, a high reduction in the total body fat mass is observed in the mice fed with the 80% HL diet. The subcutaneous and visceral adipose tissues of the mice under this diet are also significantly reduced.

It was also observed that the ingestion of hydrolysate H1 reduced the size of the adipocytes of the subcutaneous and epididymal adipose tissues. This effect was not observed on the visceral adipose tissue.

The adiposity index (the ratio between the body fat mass and the weight of the carcass of the animals) was also calculated (FIG. 16). This increases significantly after the ingestion of an HL diet compared with the ingestion of a control diet.

The adiposity index is significantly reduced in mice ingesting the 80% NL and 80% HL diets compared with the respective control diets, HL and NL, that is to say without H1 hydrolysate. The adiposity index tends to decrease in animals fed with the 50% diets of H1 in the protein energy contribution.

During this study, it was also observed that the liver of the animals that ingested the HL regime is larger than in the control mice. The addition of H1 hydrolysate in the diets causes a significant reduction in the weight of the liver whether it be in the ingestion of an NL control diet, or an HL diet.

### EXAMPLE 5

### Biological Activities of Fish Protein Hydrolysates Obtained by Means of an Enzyme Derived from Bacillus licheniformis or a Mixture of Enzymes Derived from Bacillus amyloliquefaciens and Bacillus licheniformis

For the purpose of enriching its collection of functional peptides having an activity on weight, the applicant company continued its research by testing for such an activity of the peptides obtained by enzymatic digestion using enzymes other than that derived from Bacillus subtilis.

Thirty two male Wistar rates weighing 344±4 g were divided into 4 groups of 8 rats called TMP, fish protein, peptide A, peptide P. The energy contribution of each diet was achieved by 55% proteins, 30% lipids and 15% carbohydrates. They were housed individually in grille cages and placed in a thermostatically controlled room (22°±1° C.) in a day-night cycle, knowing that rats feed at night.

The rats became habituated, over one week, to a lipidic normo-protein P14 diet containing 14% total milk proteins (TMPs), 56% carbohydrates and 30% lipids. They were fed ad libitum. After a week of habituation and once the required weight was achieved, the rats were fed daily for 21 days with their experimental diet. The various groups received the diet corresponding to their name.

TMP: Total Milk Proteins

Fish protein: natural marine source of proteins issuing from a lean fish, with white flesh.

Peptide A: peptide obtained by a method for the enzymatic hydrolysis of blue whiting using Alcalase® (enzyme derived from Bacillus licheniformis).

Peptide P: peptide obtained by a method for the enzymatic hydrolysis of blue whiting using Protamex® (enzyme derived from Bacillus amyloliquefaciens and Bacillus licheniformis).

Weighing was carried out daily together with an evaluation of the consumption of powder in grams.

Change in Weight

FIG. 17 illustrates the total gain in weight at day 21 of the rats fed ad libitum with the diets tested (the values correspond to the average±the standard deviation, n=8). The groups that do not have the same letters are statistically different (p<0.05).

After a week of habituation to the experimental conditions, the weight of the groups is not different when they are fed with the P14 reference diet (348±5 g). At the end of the experiment, the total weight gain does not show any significant difference between the Peptide groups (peptide A and peptide P) and the TMP and Fish Protein groups.

Food Intake

During the habituation period, each group has the same food intake as with the reference diet (337±4 kg). When the rats are then fed with their experimental diet, a reduction in the food intake is observed for the four groups compared with their habituation period but this reduction is not significant between the various Peptide and reference groups (FIG. 18). The groups that receive the blue whiting protein hydrolysates obtained using Protamex® and Alcalase® enzymes did not ingest any more food.

As there is no difference between the peptide A and peptide P groups and the reference groups (TMP, fish protein) concerning the gain in weight, it was concluded that peptides A and P had no effect on weight. Consequently the research work was not continued with these two hydrolysates.

1.-16. (canceled)

17. Non-therapeutic use of a fish protein hydrolysate obtained by enzymatic hydrolysis of at least protein source chosen from the group composed of the following fish: Micromesistius poutassou, Clupea harengus, Scomber scombrus, Sardina pilchardus, Trisopterus esmarki, Trachurus spp., Gadus morhua, Pollachius virens, Melanogrammus aeglefinus, Coryphaenoides rupestris, fish belonging to the order Siluriformes, said enzymatic hydrolysis being performed by an endopeptidase enzyme derived from Bacillus subtilis, for causing weight loss or the limitation or inhibition of weight gain without inducing any limitation to the food intake or any phenomenon of satiety.

18. Use according to claim 17, in which the inducing of weight loss and the limitation or inhibition of weight gain include a reduction in the body mass, in particular a reduction in the fat mass, an improvement in the body fat mass/lean body mass ratio, a reduction in adiposity, an improvement in the body composition, a regulation of the lipid metabolism, and more particularly still a stimulation of lypolysis and/or an inhibition of lypogenesis.

19. Use according to claim 17, in which the fish protein hydrolysate has:
the following molecular profile distribution: 33% to 39% molecules with a molecular weight of less than 300 Da, 34% to 37% molecules the molecular weight of which is between 300 and 1000 Da, 21% to 24% molecules the molecular weight of which is between 1000 and 3000 Da, 3% to 4% molecules the molecular weight of which is between 3000 and 5000 Da and 1% to 2% molecules the molecular weight of which is between 5000 and 10,000 Da,
a lipid content of less than 1%, by percentage of raw product,
a lucid content of less than 4%, by percentage of raw product,
a protein content above 80%, by percentage of raw product,
a mineral matter content of between 5% and 10%, by percentage of raw product.

20. Use according to claim 17, in which the fish protein hydrolysate is obtained by an obtaining method comprising:
the grinding of at least one source of proteins chosen from the group composed of the fish species Micromesistius poutassou, Clupea harengus, Scomber scombrus, Sardina pilchardus, Trisopterus esmarki, Trachurus spp., Gadus morhua, Pollachius virens, Melanogrammus aeglefinus, Coryphaenoides rupestris, of fish belonging to the order Siluriformes, in the presence of water, so as to recover the pump from the said fish,
the enzymatic hydrolysis of the said protein source at a temperature of between 50° and 75° C., for 1 to 5 hours, after the addition of an endopeptidase enzyme derived from Bacillus subtilis so as to obtain a reaction mixture,
stoppage of the said enzymatic hydrolysis by inactivation of the said enzymes after increasing the temperature of the said reaction mixture to a level not below 70° C., for 8 to 20 minutes,
separation of the protein hydrolysate obtained from the rest of the reaction mixture.

21. Use according to claim 17, in which the said enzymatic hydrolysis is carried out in an enzyme scope protein source ratio between 0.01% and 2%, preferentially equal to 0.75%, and at a hydrolysis temperature of 55° C.

22. Method of therapeutic treatment and/or prevention of excessive weight and/or illnesses related to excessive weight without inducing any limitation to the food intake or any phenomenon of satiety, characterised in that it consists of orally administering a fish protein hydrolysate obtained by enzymatic hydrolysis of at least one protein source chosen from the group composed of the fish Micromesistius poutassou, Clupea harengus, Scomber scombrus, Sardina pilchardus, Trisopterus esmarki, Trachurus spp., Gadus morhua, Pollachius virens, Melanogrammus aeglefinus, Coryphaenoides rupestris, fish belonging to the order Siluriformes, the said enzymatic hydrolysis being carried out by an endopeptidase enzyme derived from Bacillus subtilis.

23. Method according to claim 22, characterised in that the treatment and/or prevention of excessive weight comprises the induction of weight loss, the limitation and/or inhibition of weight gain, in particular reduction in fat mass, improvement to the fat mass/thin mass ratio, the treatment of adiposity, the regulation of lipid metabolism, stimulation of lypolysis and/or inhibition of lypogenesis.

24. Method according to claim 22, characterised in that the excess weight is related to obesity, to general metabolism disorders or to weight disorders symptomatic of the menopause.

25. Method according to claim 22, characterised in that the fish protein hydrolysate is as defined in claims 17 to 21.

26. Non-therapeutic treatment method for inducing weight loss, or the limitation or inhibition of weight gain, characterised in that it consists of orally administering a fish protein hydrolysate as defined in claims 17 to 21.
